(19) United States
(12) Patent Application Publication
(10) Pub. No.: US 2025/0134606 A1
(43) Pub. Date: May 1, 2025

(54) ROBOTIC SURGERY SYSTEM WITH USER INTERFACING

(71) Applicant: ORTHOSOFT ULC, Montreal (CA)

(72) Inventor: Emile ROUILLARD, Montreal (CA)

(21) Appl. No.: 18/924,039

(22) Filed: Oct. 23, 2024

Related U.S. Application Data
(60) Provisional application No. 63/592,943, filed on Oct. 25, 2023.

(51) Int. Cl. A61B 34/30; A61B 17/00; A61B 34/20; A61B 90/00

(52) U.S. Cl. CPC A61B 34/30; A61B 17/00; A61B 2017/00725; A61B 2034/2055; A61B 2090/3937

(57) ABSTRACT

A system for tracking calibrating an instrument in a robotized computer-assisted surgery, may have a processing unit; and a non-transitory computer-readable memory communicatively coupled to the processing unit and comprising computer-readable program instructions executable by the processing unit for: tracking the instrument optically as maneuvered by a robot arm in a calibration sequence; comparing optical tracking values from the calibration sequence with robot arm maneuvering data; and calibrating the instrument from the comparing for subsequent use of the instrument to perform actions on the bone.

### CROSS-REFERENCE TO RELATED APPLICATION

The present application claims the priority of U.S. Patent Application No. 63/592,943, filed on Oct. 25, 2023, the entire content of which is incorporated herein by reference.

### TECHNICAL FIELD

The present application relates to robotized computer-assisted surgery including bone and tool tracking, and to the calibration of instruments in the context of computer-assisted surgery.

### BACKGROUND OF THE ART

Tracking of surgical instruments or tools is an integral part of computer-assisted surgery (hereinafter “CAS”), including robotized CAS. The end effector, the tools, bodily parts are tracked for position and/or orientation using computerized components in such a way that relative navigation information pertaining to bodily parts is obtained. The information is then used in various interventions (e.g., orthopedic surgery, neurological surgery) with respect to the body, such as bone alterations, implant positioning, incisions and the like during surgery.

In robotized CAS, optical tracking is commonly used in different forms, for instance by the presence of optically-detectable trackers on the end effector and/or operating end of a robotic arm, in addition to being optionally present on the patient. For example, the optically-detectable trackers are passive retroreflective components on the robot, on tools and bones, though other types of trackers may be used. The trackers are viewed by a tracking device, such as a tracking camera (e.g., Navitracker®, a depth camera, and by triangulation the position and orientation of the tracker device is calculable to output navigation data. The robot arm may also be equipped with a tracker device.

In order to contribute to the precision and accuracy, tools (a.k.a., instruments, surgical instruments, etc) having tracker devices thereon may be calibrated intraoperatively or perioperatively. The calibration may consist in recording a geometric relation between the tracker device and the working end of the tool, such that the subsequent tracking of the tracker device enables the CAS system to output navigation data for the tool, which navigation data is associated with the working end of the tool. The working end of the tool may be a tip and/or axis of a registration pointer, the blade of a saw, the reaming end of a reamer and a rotational axis thereof, as examples among others.

Different approaches exist for the calibrating, including calibration devices that attached to a tool in a known manner, calibration sequences in which the tool is maneuvered in given ways relative to a fixed point or known landmark, etc. However, in spite of the calibration, the precision and accuracy may still be improved.

### SUMMARY

In accordance with a first aspect of the present disclosure, there is provided a system for tracking an instrument in a robotized computer-assisted surgery, comprising: a processing unit; and a non-transitory computer-readable memory communicatively coupled to the processing unit and comprising computer-readable program instructions executable by the processing unit for: tracking the instrument optically as maneuvered by a robot arm in a calibration sequence; obtaining robot arm maneuvering data during the calibration sequence; comparing optical tracking values from the calibration sequence with the robot arm maneuvering data; and calibrating the instrument from the comparing for subsequent use of the instrument to perform actions on the bone; and tracking the instrument optically after the calibrating.

Further in accordance with the first aspect, for instance, the calibration sequence is preprogrammed.

Still further in accordance with the first aspect, for instance, tracking the instrument optically after the calibrating is performed as the instrument is maneuvered by the robot arm.

Still further in accordance with the first aspect, for instance, calibrating the instrument from the comparing includes recording a geometrical relation between an optical tracker and a working end of the instrument.

Still further in accordance with the first aspect, for instance, calibrating the instrument from the comparing includes correcting a geometrical relation between an optical tracker and a working end of the instrument.

Still further in accordance with the first aspect, for instance, the optical tracker is included.

Still further in accordance with the first aspect, for instance, the instrument has a multifaceted tracker thereon.

Still further in accordance with the first aspect, for instance, calibrating the instrument from the comparing includes recording a geometrical relation between at least two sets of optical elements in the multifaceted tracker.

Still further in accordance with the first aspect, for instance, tracking the instrument optically after the calibrating includes tracking the instrument using a first of the at least two sets of optical elements, and switching to tracking the instrument using a second of the at least two sets of optical elements when a line of sight between the first of the at least two sets of optical element and a tracking device is disrupted.

Still further in accordance with the first aspect, for instance, calibrating the instrument from the comparing includes correcting a geometrical relation value between at least two sets of optical elements in the multifaceted tracker.

Still further in accordance with the first aspect, for instance, the multifaceted tracker is included, the multifaceted tracker having at least two sets of three optical elements.

Still further in accordance with the first aspect, for instance, tracking the instrument optically after the calibrating includes tracking the instrument using a first of the at least two sets of optical elements, and switching to tracking the instrument using a second of the at least two sets of optical elements when a line of sight between the first of the at least two sets of optical element and a tracking device is disrupted.

Still further in accordance with the first aspect, for instance, obtaining robot arm maneuvering data during the calibration sequence includes obtaining the robot arm maneuvering data from a robot controller.

Still further in accordance with the first aspect, for instance, obtaining robot arm maneuvering data during the calibration sequence includes obtaining the robot arm maneuvering data generated by sensors in the robot arm.

Still further in accordance with the first aspect, for instance, obtaining robot arm maneuvering data during the calibration sequence includes obtaining the robot arm maneuvering data from a calibration sequence file.

Still further in accordance with the first aspect, for instance, the system tracks the instrument using the robot arm maneuvering data when tracking the instrument optically is disrupted.

Still further in accordance with the first aspect, for instance, the calibration sequence is repeated and calibrating occurs at least a second time.

Still further in accordance with the first aspect, for instance, an optical tracking device is included.

### DESCRIPTION OF THE DRAWINGS

FIG. 1 is a schematic view of a robotic surgery system in accordance with an aspect of the present disclosure, relative to a patient;

FIG. 2 is a block diagram of the tracking system for robotized computer-assisted surgery of FIG. 1;

FIG. 3 is a perspective view of a tracker device that may be calibrated for use with the robotic surgery system;

FIG. 4 is a flow chart of a method for calibrating a tracker device with a robotic surgery system; and

FIG. 5 is a graph illustrating a potential inaccuracy in tracking, which inaccuracy may be corrected using the tracking system for robotized computer-assisted surgery of FIG. 1.

### DETAILED DESCRIPTION

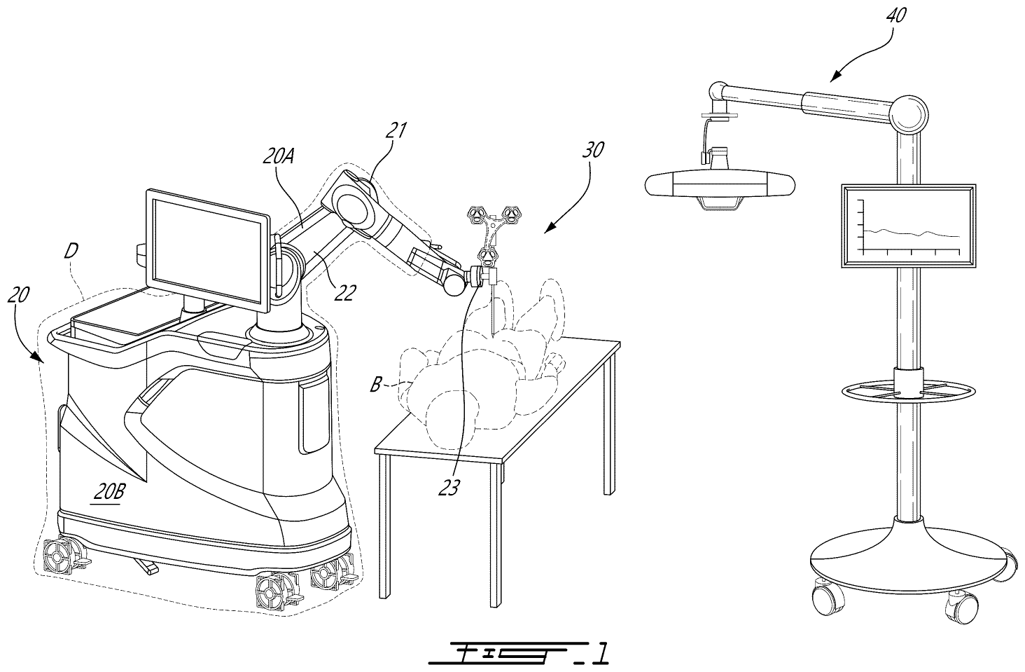

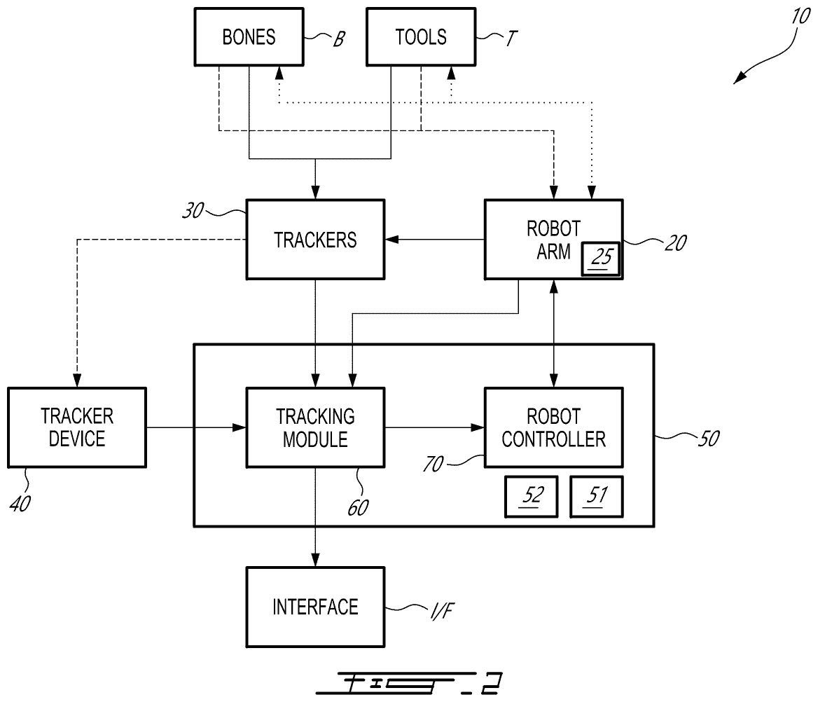

Referring to FIGS. 1 and 2, a robotic surgery system for computer-assisted surgery (CAS) system is generally shown at 10, and is used to provide surgery assistance to an operator. For simplicity, it will be referred to herein as the system 10. In FIG. 1, the system 10 is shown relative to a dummy patient in prone decubitus, but only as an example. The system 10 could be used for any body parts, including non-exhaustively hip joint, spine, and shoulder bones, for orthopedic surgery, but could also be used in other types of surgery. For example, the system 10 could be used for surgery of all sorts, such as brain surgery, and soft tissue surgery.

The robotic surgery system 10 may be robotized in a variant, and has, may have or may be used with a robot 20, optical trackers 30, a tracker device 40, a CAS controller 50 (also known as a super controller 50), a tracking module 60, and a robot controller 70 (also known as a robot driver), or any combination thereof:
The robot 20, shown by its robot arm 20A may optionally be present as the working end of the system 10, and may be used to perform or guide bone alterations as planned by an operator and/or the CAS controller 50 and as controlled by the CAS controller 50. The robot arm 20A may also be configured for collaborative/cooperative mode in which the operator may manipulate the robot arm 20, or the tool supported by the robot arm 20, though the tool may be operated by a human operator. For example, the tooling end, also known as end effector, may be manipulated by the operator while supported by the robot arm 20A. The robot 20 may be the coordinate measuring machine (CMM) of the robotic surgery system 10;
The optical trackers 30 are positioned on the robot 20, on patient tissue (e.g., bones B), and/or on the tool(s) T and surgical instruments, and provide tracking data for the robot 20, the patient and/or tools.

The tracking device 40, also known as a sensor device, apparatus, etc performs optical tracking of the optical trackers 30, so as to enable the tracking in space (a.k.a., navigation) of the robot 20, the patient and/or tools;
The CAS controller 50, also known as the super controller, includes the processor(s) and appropriate hardware and software to run a computer-assisted surgery procedure in accordance with one or more workflows. The CAS controller 50 may include or operate the tracking device 40, the tracking module 60, and/or the robot controller 70. As described hereinafter, the CAS controller 50 may also drive the robot arm 20A through a planned surgical procedure;

The tracking module 60 is tasked with determining the position and/or orientation of the various relevant objects during the surgery procedure, such as the end effector of the robot arm 20, bone(s) B and tool(s) T, using data acquired by the tracking device 40 and by the robot 20, and/or obtained from the robot controller 70. The position and/or orientation may be used by the CAS controller 50 to control the robot arm 20A;

The robot controller 70 is tasked with powering or controlling the various joints of the robot arm 20A, based on operator demands or on surgery planning, and may also be referred to as a robot controller module that is part of the super controller 50. The robot controller 70 may also optionally calculate robot movements of the robot arm 20A, so as to control movements of the robot arm 20A autonomously in some instances, i.e., without intervention from the CAS controller 50;

An additional camera(s) may be present, for instance as a complementary registration tool. The camera may for instance be mounted on the robot 20A, such as on the robot arm, such that the point of view of the camera is known in the frame of reference, also known as the coordinate system.

Other components, devices, systems, may be present, such as surgical instruments and tools T, interfaces I/F such as displays, screens, computer station, servers, and like etc. Secondary tracking systems may also be used for redundancy.

Referring to FIG. 1, the robot 20 may have the robot arm 20A stand from a base 20B, for instance in a fixed relation relative to the operating-room (OR) table supporting the patient, whether it is attached or detached from the table. The robot arm 20A has a plurality of joints 21 and links 22, of any appropriate form, to support an end effector 23 that may interface with the patient, or may be used during surgery without interfacing with the patient. For example, the end effector or tool head may optionally incorporate a force/torque sensor for collaborative/cooperative control mode, in which an operator manipulates the robot arm 20A. The robot arm 20A is shown being a serial mechanism, arranged for the tool head 23 to be displaceable in a desired number of degrees of freedom (DOF). The tool head 23 may for example be a support that is not actuated, the support being used to support a tool, with the robot arm 20A used to position the tool relative to the patient. In a variant, the robot arm 20A controls 6-DOF movements of the tool head, i.e., X, Y, Z in the coordinate system, and pitch, roll and yaw. Fewer or additional DOFs may be present. For simplicity, only a fragmented illustration of the joints 21 and links 22 is provided, but more joints 21 of different types may be present to move the end effector 23 in the manner described above. The joints 21 are powered for the robot arm 20A to move as controlled by the CAS controller 50 in the six DOFs, and in such a way that the position and orientation of the end effector 23 in the coordinate system may be known, for instance by readings from encoders on the various joints 21. Therefore, the powering of the joints is such that the end effector 23 of the robot arm 20A may execute precise movements, such as moving along a single direction in one translation DOF, or being restricted to moving along a plane, among possibilities. Such robot arms 20A are known, for instance as described in U.S. patent application Ser. No. 11/610,728, and incorporated herein by reference.

The end effector 23 of robot arm 20A may be defined by a chuck or like tool interface, typically actuatable in rotation. As a non-exhaustive example, numerous tools may be used as end effector for the robot arm 20, such tools including a registration pointer as shown in FIG. 1, equipped with a tracker device 30, a reamer (e.g., cylindrical, tapered), a reciprocating saw, a retractor, a camera, an ultrasound unit, a laser rangefinder or light-emitting device (e.g., the indicator device of U.S. Pat. No. 8,882,777), a laminar spreader, an instrument holder, or a cutting guide, depending on the nature of the surgery. The various tools may be part of a multi-mandible configuration or may be interchangeable, whether with human assistance, or as an automated process. The installation of a tool in the tool head may then require some calibration in order to track the installed tool in the X, Y, Z coordinate system of the robot arm 20.

The end effector 23 of the robot arm 20A may be positioned by the robot 20 relative to surgical area A in a desired orientation according to a surgical plan, such as a plan based on preoperative imaging. Due to the proximity between the robot 20 and the surgical area A, the robot 20 may be covered partially with a surgical drape D, also known as a surgical robotic drape. The surgical drape D is a sterile panel (or panels), tubes, bags or the like that form(s) a physical barrier between the sterile zone (e.g., surgical area) and some equipment that may not fully comply with sterilization standards, such as the robot 20. In an embodiment, the surgical drape D is transparent such that one can see through the drape D. In an embodiment, the robot is entirely covered with the surgical drape D, and this includes the base 20B, but with the exception of the end effector 23. Indeed, as the end effector 23 interacts or may interact with the human body, it may be sterilized and may not need to be covered by the surgical drape D, to access the patient. Some part of the robot 20 may also be on the sterile side of the surgical drape D. In a variant, a portion of the robot arm 20 is covered by the surgical drape D. For example, the surgical drape D may be in accordance with U.S. patent application Ser. No. 15/803,247, filed on Nov. 3, 2017 and incorporated herein by reference.

In order to position the end effector 23 of the robot arm 20A relative to the patient B, the CAS controller 50 can manipulate the robot arm 20A automatically (without human intervention), or by a surgeon manually operating the robot arm 20A (e.g. physically manipulating, via a remote controller through the interface I/F) to move the end effector 23 of the robot arm 20A to the desired location, e.g., a location called for by a surgical plan to align an instrument relative to the anatomy. Once aligned, a step of a surgical procedure can be performed, such as by using the end effector 23. To assist in the maneuvering and navigating of the robot arm 20A, a tracker device 30 may optionally be secured to the distalmost link, and may be distinct from the tracker device 30 on the instrument supported by the end effector 23.

As shown in FIG. 2, the robot arm 20A may include sensors 25 in its various joints 21 and links 22. The sensors 25 may be of any appropriate type, such as rotary encoders, optical sensors, position switches that are a non-exhaustive list of potential sensors, for the position and orientation of the end effector, and of the tool in the end effector 23 to be known. More particularly, the tracking module 60 may determine the position and orientation of the robot 20 in a frame of reference of the robot 20, such as by obtaining the position (x,y,z) and orientation (phi, theta, ro) of the end effector 23 from the CAS controller 50 using the sensors 25 in the robot arm 20A, i.e., robot coordinates may be an integrated function of the robot 20 in that it may determine the position and orientation of its end effector 23 with respect to its coordinate system. Using the data from the sensors 25, the robot 20 may be the coordinate measuring machine (CMM) of the robotic surgery system 10, with a frame of reference (e.g., coordinate system, referential system) of the procedure being relative to the fixed position of the base 20B of the robot 20. The sensors 25 must provide the precision and accuracy appropriate for surgical procedures. The coupling of tools to the robot arm 20A may automatically cause a registration of the position and orientation of the tools in the frame of reference of the robot 20, though steps of calibration could be performed, as explained below.

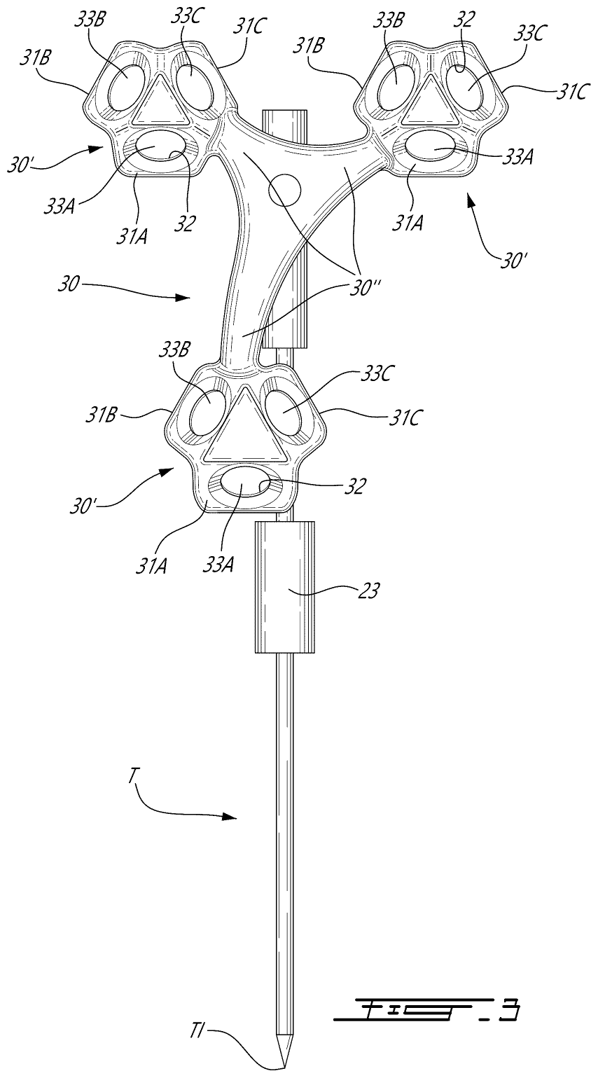

Referring to FIG. 1, an exemplary tracker 30 is shown secured to the instrument T and at the end effector 23 on the robot 20, and may also or alternatively be on the robot arm 20 and/or on the bones B. The trackers 30 may be known as trackable elements, markers, navigation markers, active sensors (e.g., wired or wireless) that may for example include infrared emitters. In a variant, the trackers 30 are passive retro-reflective elements, that reflect light. The trackers 30 have and/or may be arranged in a known geometry so as to be recognizably through detection by the tracker device 40. For example, the trackers 30 may be retro-reflective lenses. Referring to FIG. 3, an exemplary tracker 30 is shown as being mounted to a registration pointer T having a tip T1. This is one of numerous tools that may be equipped with the tracker 30. The tracker 30 of FIG. 3, may be as described in U.S. Pat. No. 8,386,022.

The tracker 30 may thus be known as a multifaceted tracker. The tracker 30 of the exemplary embodiment has three tracker ends 30′ supported by arms 30″ that interface the tracker ends 30′ to the instrument T. Each tracker end 30′ is provided in three sets of three detectable elements. For example, the tracker ends 30′ are each provided with a pyramidal body having faces 31A, 31B and 31C (hereinafter faces 31 unless otherwise indicated). The faces 31 each define an opening 32 having a given geometrical shape. In the embodiment of FIG. 3, the given geometrical shape is a circle.

Retro-reflective surfaces are positioned in the openings 32, so as to form circular optical elements 33A, 33B, and 33C, respectively provided in the faces 31A, 31B, and 31C of the tracker ends 30′. Other shapes are also considered for the optical elements 33. The retro-reflective surfaces are made of a retro-reflective material that will be detected by the optical tracker device 40 associated with the CAS system 10. For instance, the material Scotch Lite™ is suited to be used as retro-reflective surface.

As the optical elements 33 must be in a given geometrical pattern to be recognized by the optical tracker device 40 of the CAS system 10, the optical elements 33 are regrouped in one embodiment in sets of three. Referring to FIG. 1, a first set of three elements 33 consists of the optical elements 33A, each of which is in a different one of the tracker ends 30′. Similarly, a second set consists of the elements 33B, and a third set consists of the elements 33C. There may be a single set of elements 33, a pair of sets of elements 33, or more than the three sets of elements 33 illustrated.

In the embodiment of FIG. 3, each of the elements of a same set (e.g., the first set of elements 33A) are parallel to a same plane, though this exact geometrical relation may not be necessary. Accordingly, the elements 33A are visible from a same field of view. The sets of elements 33 are strategically positioned with respect to one another so as to optimize a range of visibility of the tracker device 10. More specifically, the sets are positioned such that once the tracker device 40 of the CAS system 10 loses sight of one of the sets, another set is visible. This ensures the continuous tracking of the tool T having a tracker device 30 within a given range of field of view.

The sets each form a geometrical pattern that is recognized by the tracking module 60 of the CAS system 10. The combination of circular openings 32 and retro-reflective surface gives a circular shape to the optical elements 33. According to the angle of view of the tracker device 40, these circles will not always appear as being circular in shape. Therefore, the position of the center of the circles can be calculated as a function of the shape perceived from the angle of view by the optical sensor apparatus.

In the embodiment of FIG. 3, the geometrical pattern therefore consists of a triangle defined by the centers of the optical elements 33 of the sets. It is suggested that the three triangles of the three different sets of optical elements 33 be of different shape, with each triangle being associated with a specific orientation with respect to the tool. Alternatively, the three triangles formed by the three different sets may be the same, but the perceived shape of the circular reflective surfaces 33 must be used to identify which of the three sets of reflective surfaces 33 is seen. There may be more or less optical elements, and sets of optical elements, as described in U.S. Pat. No. 8,386,022. Moreover, although triangular geometrical patterns are illustrated, it is contemplated to use other geometrical patterns, such as lines and various polygonal shapes.

It is pointed out that a calibration of the surgical tool with the tracker 30 of FIG. 3, as any other tracker thereon, is preferably performed prior to the use of the tool T, to calibrate a position and/or orientation of each of the detectable geometrical patterns with respect to the tool T, in the manner explained below. In order to optimize the range of visibility of the tracker device 10, the arrangement of the circular optical elements 33 on a tracker end 30′ is taken into consideration.

In an embodiment, the trackers 30 may be active emitters. The trackers 30 may also be retroreflective spheres, QR tokens, etc, the embodiment of FIG. 3 merely provided as an example.

In FIGS. 1 and 2, the tracker device 40 is shown as being embodied by an image capture device, capable of illuminating its environment. In a variant, the tracker device 40 may have two (or more) points of view, such that triangulation can be used to determine the position of the tracker devices 30 in space, i.e., the coordinate system of the robotic surgery system 10. The tracker device 40 may emit light, or use ambient light, to observe the trackers 30 from its points of view, so as to determine a position of the trackers 30 relative to itself. By knowing the geometry of the arrangements of trackers 30, the tracker device 40 can produce navigation data enabling the locating of objects within the coordinate system of the robotic surgery system 10. In an embodiment, the tracker device 40 is of the type known as the Polaris products by Northern Digital Inc. The tracker device 40 may form the complementary part of the CMM function of the robotic surgery system 10, with the trackers 30 on the robot base 20A for example.

Referring to FIG. 2, the CAS controller 50 is shown in greater detail relative to the other components of the robotic surgery system 10. The CAS controller 50 has a processor unit 51 and a non-transitory computer-readable memory 52 communicatively coupled to the processing unit 51 and configured for executing computer-readable program instructions executable by the processing unit 51 to perform some functions, such as tracking the patient tissue and tools, using the position and orientation data from the robot 20 and the readings from the tracker device 40. Accordingly, as part of the operation of the CAS controller 50, the computer-readable program instructions may include an operating system that may be viewed by a user or operator as a GUI on one or more of the interfaces of the robotic surgery system 10. It is via this or these interfaces that the user or operator may interface with the robotic surgery system, be guided by a surgical workflow, obtain navigation data, etc. The CAS controller 50 may also control the movement of the robot arm 20A via the robot controller module 70. The robotic surgery system 10 may comprise various types of interfaces I/F, for the information to be provided to the operator. The interfaces I/F may include and/or screens including wireless portable devices (e.g., phones, tablets), audio guidance, LED displays, head-mounted display for virtual reality, augmented reality, mixed reality, among many other possibilities. For example, the interface I/F comprises a graphic-user interface (GUI) operated by the system 10. The CAS controller 50 may also display images captured pre-operatively, or using cameras associated with the procedure (e.g., 3D camera, laparoscopic cameras, tool mounted cameras), for instance to be used in the collaborative/cooperative control mode of the system 10, or for visual supervision by the operator of the system 10, with augmented reality for example. The CAS controller 50 may drive the robot arm 20A, in performing the surgical procedure based on the surgery planning achieved pre-operatively, or in maintaining a given position and orientation to support a tool. The CAS controller 50 may run various modules, in the form of algorithms, code, non-transient executable instructions, etc, in order to operate the robotic surgery system 10 in the manner described herein. The CAS controller 50 may be part of any suitable processor unit, such as a personal computer or computers including laptops and desktops, tablets, server, etc.

The tracking module 60 may be a subpart of the CAS controller 50, or an independent module or system. The tracking module 60 receives the position and orientation data from the robot 20 and the readings from the tracker device 40. The tracking module 60 may hence determine the relative position of the objects relative to the robot arm 20A in a manner described below. The tracking module 60 may also be provided with models of the objects to be tracked. For example, the tracking module 60 may track bones and tools, and hence may use virtual bone models and tool models. The bone models may be acquired from pre-operative imaging (e.g., MRI, CT-scans), for example in 3D or in multiple 2D views, including with 2D X-ray to 3D bone model technologies. The virtual bone models may also include some image processing done preoperatively, for example to remove soft tissue or refine the surfaces that will be exposed and tracked. The virtual bone models may be of greater resolution at the parts of the bone that will be tracked during surgery, such as the knee articulation in knee surgery. The bone models may also carry additional orientation data, such as various axes (e.g., longitudinal axis, mechanical axis, etc). The bone models may therefore be patient specific. It is also considered to obtain bone models from a bone model library, with the data obtained from the video images used to match a generated 3D surface of the bone with a bone from the bone atlas. The virtual tool models may be provided by the tool manufacturer, or may also be generated in any appropriate way so as to be a virtual 3D representation of the tool(s).

Additional data may also be available, such as tool orientation (e.g., axis data and geometry). By having access to bone and tool models, the tracking module 60 may obtain additional information, such as the axes related to bones or tools.

Still referring to FIG. 2, the CAS controller 50 may have the robot controller 70 integrated therein. However, the robot controller 70 may be physically separated from the CAS controller 50, for instance by being integrated into the robot 20 (e.g., in the robot base 20B). The robot controller 70 is tasked with powering and/or controlling the various joints of the robot arm 20A. The robot controller 70 may also optionally calculate robot movements of the robot arm 20A, so as to control movements of the robot arm 20A autonomously in some instances, i.e., without intervention from the CAS controller 50. There may be some force feedback provided by the robot arm 20A to avoid damaging the bones, to avoid impacting other parts of the patient or equipment and/or personnel. The robot controller 70 may perform actions based on a surgery planning. The surgery planning may be a module programmed specifically for any given patient, according to the parameters of surgery desired by an operator such as an engineer and/or surgeon. The parameters may include geometry of selected, planned bone cuts, planned cut depths, sequence or workflow of alterations with a sequence of surgical steps and tools, tools used, etc.

As observed herein, the trackers 30/the tracker device 40 and the tracking from the robot controller 70 may be complementary and/or redundant tracking technologies. The position and orientation of the surgical tool T calculated by the tracking module 60 using optical tracking (i.e., 30 and 40) may be redundant over the tracking data provided by the robot controller 70 and/or the CAS controller 50 and its embedded robot arm sensors 25, referred to as maneuvering data for the robot arm 20A. However, the redundancy may assist in ensuring the accuracy of the tracking of the surgical tool T, and end effector 23. More particularly, the combination of the navigation data from the tracker device 40 and that from robot controller 70 may strategically be used to improve the accuracy of the calibration of the instruments T with their trackers 30. The present system 10 and related method may apply to the instruments T with trackers 30 as in FIG. 3, but also to other types of trackers 30, such as retroreflective spheres, QR codes, etc. While the tracker 30 of FIG. 3 may advantageously be used as a cost-effective and robust solution within allowable accuracy tolerances, there may be in some instances the possibility to further improve the accuracy of the tracking with the tracker 30 of FIG. 3, via the calibration approach described herein. Such calibration approach uses the navigation data obtained from the optical tracking, e.g., the tracker(s) 30 and the tracker device 40, and also uses the maneuvering data, i.e., data from the robot controller 70 from robot arm control commands and/or from feedback from the robot arm 20A, such as via sensors 25 (e.g. encoders in the joints and/or motors of the robot arm 20A.

With the instrument T and tracker 30 secured to the end effector 23 of the robot arm 20A, with the end effector 23's movements being tracked using its own tracker 30, such as in the manner shown in FIG. 1, the CAS system 10 optically tracks the trackers 30. If a tracker 30 is on the robot arm 20A, the CAS system 10 may track movements of the end effector 23, to isolate movements of the instrument T. In a variant, the robot arm 20A then performs a calibration sequence, i.e., moves the robot arm 20A, such that the movements span a given range of distance and/or include translations and rotations and/or include contact of the instrument and/or the tracker 30 with a reference object. As a possibility, the calibration sequence is pre-programmed and known by the CAS controller 50, and thus the maneuvering data is accessible to the CAS controller 50. It may also be possible that the maneuvering data is updated or recorded using the feedback from the sensors 25 in the robot arm 20A, or control commands of the robot controller 70, such as if the calibration sequence 70 is achieved by human input (e.g., joystick or like interface, collaborative mode in which forces are applied to the robot arm 20A). The CAS controller 50 may have the geometrical relation between the tool T (e.g., its tip T1) and the tracker 30 thereon, such that the geometrical relation is used in the calibration.

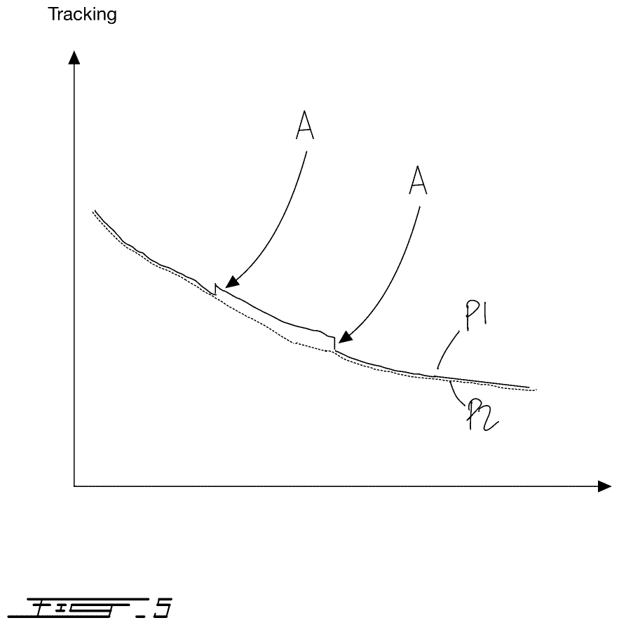

The CAS controller 50 may then compare optical tracking values from the calibration sequence with the maneuvering data of the robot arm 20A. As it may be anticipated that the maneuvering data of the robot arm 20A may have a greater accuracy, any difference from the comparison between optical tracking values with the maneuvering data may result in a correction of the calibration file, such as by determining the corrective values from the differences. Notably, when the tracking switches from one set 33 to another, for the tracker 30 on the tool T and/or the tracker 30 on the robot arm 20A, some inaccuracy in reading may occur, which inaccuracy may have an impact on the calibration file. This is described for instance with reference to FIG. 5 below. For example, the calibration file may include a geometrical relation (e.g., position and orientation) between the working end of the instrument T and the tracker 30 (whether acquire during calibration tracking or being preprogrammed from tool specifications); a geometrical relation between the two or more sets 33 of a tracker 30 (e.g., set of optical elements 33A, set of optical elements 33B, etc), for instance relative to a reference point on the instrument. The geometrical relation may be adjusted, corrected, based on the difference. In a variant, there is no difference, such that the calibration file remains as it was, after the comparing. In another variant, the user may be prompted to recalibrate the instrument T and tracker 30.

Thus, if the tracker 30 is one that corresponds to the multifaceted tracker of FIG. 3, the calibration file may include a geometrical relation between the working end of the instrument T and all three of the geometrical patterns (e.g., the triangular patterns of 31A, of 31B and of 31C), but also the geometrical relation between the two or more triangular patterns of 31A, of 31B and of 31C (three shown in FIG. 3). The calibration approach may result in the corrections being made to only one of geometrical relations between the working end of the instrument T and the triangular patterns, or two of the three (if there are three), or all of them. The calibration approach may result in the corrections being made for the geometrical relation between two or more of the triangular patterns.

Thus, the calibrating of the instrument T from the comparing may optionally include making corrections in the geometrical relation between working end of the instrument T and the tracker 30, and/or between the sets of optical elements (e.g., set of 33A versus set of 33B versus set of 33C). Once calibrating is achieved, the instrument T may subsequently be used to perform actions on the bone. For example, if the instrument T is a registration pointer as in FIG. 3, surfacic data may be acquired to generate bone models of the bone. The robot arm maneuvering data is used to detect an inaccuracy, and to assist in correcting it.

The redundancy of optical navigation from the tracker device 40 and of maneuvering data for the robot controller 70 may also be used as a safeguard against incorrect tracking from the CAS controller 50, for instance due to relative movement between the robot 20, the tracker device 40, and the patient and/or table, after calibrating has been done. Also, the tracking of the tool using the tracking module 60 may be used to detect any discrepancy between a calculated position and orientation of the surgical tool T through the sensors (e.g., encoders) on the robot arm 20A, and the actual position and orientation of the surgical tool. For example, an improper mount of the tool T into the chuck of the robot arm 20A could be detected from the output of the tracking module 60, when verified by comparing the position and orientation from the CAS controller 50 (e.g., obtained from the encoders on the robot arm 20A) with the optical tracking of a tracker 30 on the end effector 23. The operator may be prompted to verify the mount, via the interface I/F or head-mounted display 20. Moreover, the redundancy may enable the use of some of the trackers 30 as user interfaces, for the user to communicate with the CAS controller 50.

Consequently, the tracking module 60 may combine the optical tracking data from the tracker device 40 to the position and orientation data from the sensors 25 embedded in the robot arm 20A, for the positional tracking data for the objects may be calculated by the tracking module 60, as detailed below. Therefore, the combination by the tracking module 60 of the tracking from the robot arm 20A and that from the tracker device 40 enable the tracking module 60 to track objects with a continuous and robust navigation data.

In an embodiment, the tracking module 60 uses a tracker 30 on the bone B or other body portion or OR table to obtain the orientation of the bone B in the coordinate system, and locates the bone B using other methods, such as obtaining the position and orientation of a probing tool using the encoders in the robot arm 20A, in a registration procedure described below. Stated differently, the bone B may be fixed on the OR table and the system 10 may rely on trackers 30 fixed to the OR table to optically track the bone B.

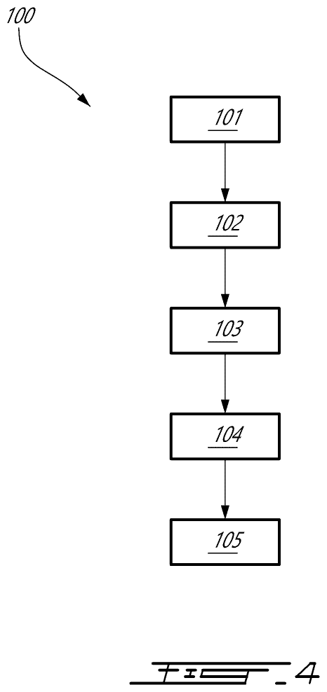

Now that the various components of the robotic surgery system 10 have been described, a contemplated procedure performed with the robotic surgery system 10 or with a similar CAS system is set forth, with reference to a flow chart 100 illustrative of a method for tracking an end effector of a robot in computer-assisted surgery is shown at in FIG. 4, and is an example of a procedure that may be performed by the CAS controller 50 and/or other parts of the robotic surgery system 10 of the present disclosure. For example, the method 100, may be computer-readable program instructions in the non-transitory computer-readable memory 52 for example, and executable by the processing unit 51 communicatively coupled to the processing unit 51.

According to 101, an instrument held by a robot arm is optically tracked as maneuvered by the robot arm in a calibration sequence. In a variant, the calibration sequence includes a predefined (e.g., programmed) set of movements of the robot arm. The robot arm may also be optically tracked.

According to 102, robot arm maneuvering data for the calibration sequence is obtained. The maneuvering data may be obtained from a calibration sequence file and/or from robot arm feedback and/or from the robot controller 70 using data from sensors in the robot arm.

According to 103, optical tracking values from the calibration sequence are compared with the robot arm maneuvering data.

According to 104, the instrument is calibrated from the comparing. This may include not making any correction to a calibration file as the comparing may indicate a match between optical tracking values and robot arm maneuvering data. 104 may also include making corrections to the geometrical relation between the tracker device 30 and the instrument T, for subsequent use in the optical tracking of the instrument T. 104 may also include making corrections to the geometrical relation between two or more sets of optical elements 33 of the tracker device 30 when the tracker device 30 is a multifaceted tracker such as that shown in FIG. 3. For example, in FIG. 5, a graph illustrates a tracking of a reference point of a tool, upon which is mounted the tracker device 30 as in FIG. 3, for example during a calibration sequence. As observed, curve P1 is illustrative of the position of the reference point as determined from optical tracking, such as by the combined actions of the tracking device 40 and the tracking module 60. It can be observed that there are steps A in the tracking, as an example of possible errors. These steps A may occur when the optical tracking switches from one set of optical elements 33 (e.g., set of optical elements 33A) to another set of optical elements 33 (e.g., set of optical elements 33B or 33C). The scale on the graph of FIG. 5 may not be representative of any magnitude of error, but indicates that there may be an inaccuracy, when the tracking device 40 switches its tracking from one of the sets 33 to another.

Also illustrated in the graph of FIG. 5 is a curve P2. The curve P2 is obtained from robotic arm sensors, referred to herein as robot arm maneuvering data. It can be observed that there are no steps A in the curve P2. Therefore, the curve P2 may be used to indicate the presence of steps A and/or to correct the curve P1 to remove the steps A. As a result, the calibration may rely on more precise tracking data.

According to 105, the instrument is tracked while being used to perform actions on the bone. 105 may also include continuously tracking the instrument with optical tracking and/or maneuvering data. 105 may also include outputting the tracking data, and this may be in the form of images on an interface, numerical data, etc. 105 is performed using the calibration file, with geometrical relations being used to determine a position and/or orientation of a working end of an instrument. The geometrical relations may have been corrected in 104. The tracking occurs in real-time or quasi real-time, i.e., the tracking values are continuously updated at a frequency that may be faster than a reaction time of a human operator, for example. Tracking the instrument T may be done using a first of the sets of optical elements 33 (e.g., 33A), and then switching to tracking the instrument using a second of the sets of optical elements 33 (e.g., 33B or 33C) when a line of sight between the first sets of optical element (e.g., 33A) and the tracking device 40 is disrupted. When visibility between the tracker 30 and the tracking device 40 is disrupted, the tracking may be done using maneuvering data from the robot controller 70.

105 may also include continuing a comparison between optical tracking data and maneuvering data to look for incoherences. The calibration sequence and calibrating (e.g., 101 to 104) may be repeated at least a second time.

Because of the redundancy of tracking, notably by the data from the sensors 25 and the data from the optical tracking, the system 10 may adjust to movement between components of the CMM, as the movement can be quantified. Accordingly, the surgical workflow may not need to pause for a complete recalibration of the robot 20 and of the patient to be done in the frame of reference. The system 10 may quantify the adjustment resulting from the relative movement of the robot 20 and/or the tracker device 40, and the surgical workflow may be continued.

Again, the distinct sources of tracking data, i.e., the embedded tracking from the sensors 25 in the robot arm 20, and optical tracking using the robot base 20A as CMM, such as through the surgical drape, and other trackers 30, ensure that sufficient tracking data is available for the tracking module 60 (FIG. 2) to determine a position of the bone B and of the end effector 23 in the frame of reference. The tracking module 60 may adjust the readings if movement is detected for the tracker device 40, with the configuration of the robotic surgery system 10.

The system 10 may thus be generally described as being for tracking an instrument in a robotized computer-assisted surgery, and may include: a processing unit; and a non-transitory computer-readable memory communicatively coupled to the processing unit and comprising computer-readable program instructions executable by the processing unit for: tracking the instrument optically as maneuvered by a robot arm in a calibration sequence; obtaining robot arm maneuvering data during the calibration sequence; comparing optical tracking values from the calibration sequence with the robot arm maneuvering data; and calibrating the instrument from the comparing for subsequent use of the instrument to perform actions on the bone; and tracking the instrument optically after the calibrating.

1. A system for tracking an instrument in a robotized computer-assisted surgery, comprising:
a processing unit; and
a non-transitory computer-readable memory communicatively coupled to the processing unit and comprising computer-readable program instructions executable by the processing unit for:
tracking the instrument optically as maneuvered by a robot arm in a calibration sequence;
obtaining robot arm maneuvering data during the calibration sequence;
comparing optical tracking values from the calibration sequence with the robot arm maneuvering data; and
calibrating the instrument from the comparing for subsequent use of the instrument to perform actions on the bone; and
tracking the instrument optically after the calibrating.

2. The system according to claim 1, wherein the calibration sequence is preprogrammed.

3. The system according to claim 1, wherein tracking the instrument optically after the calibrating is performed as the instrument is maneuvered by the robot arm.

4. The system according to claim 1, wherein calibrating the instrument from the comparing includes recording a geometrical relation between an optical tracker and a working end of the instrument.

5. The system according to claim 4, including the optical tracker.

6. The system according to claim 1, wherein calibrating the instrument from the comparing includes correcting a geometrical relation between an optical tracker and a working end of the instrument.

7. The system according to claim 6, including the optical tracker.

8. The system according to claim 1, wherein the instrument has a multifaceted tracker thereon.

9. The system according to claim 8, wherein calibrating the instrument from the comparing includes recording a geometrical relation between at least two sets of optical elements in the multifaceted tracker.

10. The system according to claim 9, including the multifaceted tracker, the multifaceted tracker having at least two sets of three optical elements.

11. The system according to claim 9, wherein tracking the instrument optically after the calibrating includes tracking the instrument using a first of the at least two sets of optical elements, and switching to tracking the instrument using a second of the at least two sets of optical elements when a line of sight between the first of the at least two sets of optical element and a tracking device is disrupted.

12. The system according to claim 8, wherein calibrating the instrument from the comparing includes correcting a geometrical relation value between at least two sets of optical elements in the multifaceted tracker.

13. The system according to claim 12, including the multifaceted tracker, the multifaceted tracker having at least two sets of three optical elements.

14. The system according to claim 12, wherein tracking the instrument optically after the calibrating includes tracking the instrument using a first of the at least two sets of optical elements, and switching to tracking the instrument using a second of the at least two sets of optical elements when a line of sight between the first of the at least two sets of optical element and a tracking device is disrupted.

15. The system according to claim 1, wherein obtaining robot arm maneuvering data during the calibration sequence includes obtaining the robot arm maneuvering data from a robot controller.

16. The system according to claim 1, wherein obtaining robot arm maneuvering data during the calibration sequence includes obtaining the robot arm maneuvering data generated by sensors in the robot arm.

17. The system according to claim 1, wherein obtaining robot arm maneuvering data during the calibration sequence includes obtaining the robot arm maneuvering data from a calibration sequence file.

18. The system according to claim 1, further including tracking the instrument using the robot arm maneuvering data when tracking the instrument optically is disrupted.

19. The system according to claim 1, further including repeating the calibration sequence and calibrating at least a second time.

20. The system according to claim 1, further including an optical tracking device.
